(19) United States
(12) Patent Application Publication
(10) Pub. No.: US 2006/0222716 A1
(43) Pub. Date: Oct. 5, 2006

(54) Colloidal solid lipid vehicle for pharmaceutical use

(76) Inventors: Joseph Schwarz, Richmond Hill (CA); Michael Weisspapir, Toronto (CA)

Correspondence Address: MCCARTHY TETRAULT LLP, BOX 48, SUITE 4700,, 66WELLINGTON STREET WEST, TORONTO, ON, M5K 1E6, CA

(21) Appl. No.: 11/197,309

(22) Filed: Aug. 5, 2005

Related U.S. Application Data
(60) Provisional application No. 60/667,069, filed on Apr. 1, 2005.

(51) Int. Cl. A61K 38/14; A61K 31/7048; A61K 31/7034; A61K 31/545; A61K 31/496; A61K 9/14

(52) U.S. Cl. 424/489; 977/906; 514/35; 514/37; 514/8; 514/192; 514/28; 514/200; 514/253.08

(57) ABSTRACT

The invention provides a drug carrier that includes a solid lipid nanoparticle (SLN), wherein the SLN includes tocopherol or a derivative thereof. The invention also provides a pharmaceutical composition that includes a SLN and a biologically active compound, wherein the SLN comprises tocopherol or a derivative thereof. The present invention further provides a colloidal drug delivery system that includes solid lipid nanoparticles (SLNs), wherein the SLNs comprise tocopherol or a derivative thereof. Also provided are methods for preparing the drug carrier, pharmaceutical composition, and colloidal drug delivery system of the invention.

### RELATED APPLICATION

This application claims the benefit of U.S. Provisional Patent Application No. 60/667,069, filed on Apr. 1, 2005, and entitled “COLLOIDAL SOLID LIPID VEHICLE FOR PHARMACEUTICAL USE”, the contents of which are hereby incorporated by reference herein.

### FIELD OF THE INVENTION

This invention relates to the field of colloidal solid lipid vehicles for pharmaceutical use.

### BACKGROUND OF THE INVENTION

Colloidal vehicles (e.g., submicron emulsions, microemulsions, liposomes, nanoparticles, nanocapsules, nanopellets, niosomes, nanocrystals, and the like), which may be loaded with biologically active compounds of different types, have been widely investigated for targeted or modified drug delivery. Particulate vehicle systems may allow for delivery of a loaded drug to a desired site of action, and may provide an optimized drug release profile (Muller and Hildebrand, Pharmazeutische Technologie: Moderne Arzneiformen (Stuttgart: Wissenschaftliche Verlagsgesellschaft, 1997). Use of particulate vehicle systems can also reduce side effects associated with drug administration.

Serious limitations are associated with the use of existing colloidal formulations for drug delivery. Oil-in-water (O/W) emulsions cannot be loaded with water-soluble compounds. Moreover, O/W emulsions cannot provide a prolonged release, because the active ingredient, which is dissolved in the emulsion drops, redistributes itself into the aqueous blood phase within milliseconds upon dilution (e.g., upon injection into the blood) (C. Washington, in Emulsions and Nanosuspensions for the Formulation of Poorly Soluble Drugs, Muller et al., eds. (Stuttgart: Medpharm Scientific Publishers, 1998), 101-117). Use of these colloidal systems is also limited by the need for complex equipment, such as high-pressure homogenizers, microfluidizers, or instruments for prolonged sonication.

Microemulsions show pronounced hematolytic behavior, due to the high content of surfactants. By way of example, U.S. Pat. No. 6,419,949 to Gasco (“Microparticles for drug delivery across mucosa and the blood-brain barrier”) discloses an aqueous dispersion of microparticles comprising stearic acid and an antibiotic. The solid lipid nanoparticles (SLNs) are obtained by precipitation of the lipid nanoparticles from a warm microemulsion containing the drug, a stearate, a phospholipid, and sodium taurocholate, subsequent to dilution with cold water, followed by ultrafiltration. Materials used for the preparation of polymeric nanoparticles, such as cyanoacrylates or lactic and glycolic polymers, are usually associated with cytotoxicity, and drug loading for nanoparticles is also limited.

Liposomes are efficient for inclusion of water-soluble drugs in the internalized phase and hydrophobic molecules inside bilayers. For example, U.S. Pat. No. 5,188,837 to Domb (“Lipospheres for controlled delivery of substances”) describes the preparation of slowly degradable spherical particles of 5-500 microns for extended drug delivery. A microsuspension containing lipospheres, which are solid, water-insoluble microparticles, each having a layer of phospholipid embedded on its surface, are also described.

Despite their advantages, as described above, liposomes have poor stability properties. Furthermore, a prolonged release from liposomes is possible only to a limited extent, because identical redistribution processes of the active ingredient, and the metabolization of the phospholipids of the liposomes, limit the release time. The preparation of liposomes is also typically based on the use of toxic organic solvents, such as chloroform, and it may be difficult to eliminate the solvent completely.

Solid lipid nanoparticles are particles made from solid lipids. They represent an alternative carrier system to traditional colloidal carriers, such as emulsions and liposomes (Muller et al., Solid lipid nanoparticles (SLN) for controlled drug delivery: a review of the state of the art. Eur. J. Pharm. Biopharm., 50(1):161-177, 2000).

U.S. Pat. No. 5,576,016 to Amselem et al. (“Solid fat nanoemulsions as drug delivery vehicles”) describes the use of fatty triglycerides as a basis for SLNs. Additionally, U.S. Pat. No. 5,989,583 to Amselem (“Solid lipid compositions of lipophilic compounds for enhanced oral bioavailability”) discloses multilayer compositions, each comprising a fat core coated with multiple layers of phospholipid (Emulsomes®).

U.S. Pat. No. 6,551,619 to Penkler et al. (“Pharmaceutical cyclosporin formulation with improved biopharmaceutical properties, improved physical quality and greater stability, and method for producing said formulation”) describes a method for the preparation of triglyceride-based SLNs using high-pressure homogenization. The SLNs are loaded with cyclosporin, and are stabilized with ionic or non-ionic surfactants.

U.S. Pat. No. 6,197,349 to Westesen et al. (“Particles with modified physicochemical properties, their preparation and uses”) describes SLNs comprising supercooled molten glycerides. Similarly, U.S. Pat. Nos. 5,885,486 and 6,207,178 to Westesen et al. (“Solid lipid particles, particles of bioactive agents and methods for the manufacture and use thereof”) disclose highly stable triglyceride-based SLNs, loaded with various hydrophobic drugs.

U.S. Pat. No. 6,770,299 to Muller (“Lipid matrix-drug conjugates particle for controlled release of active ingredient”) describes SLNs comprising lipid-drug conjugates (LDC) which are linked via covalent bonds, electrostatic interactions, dipole moments, dispersion forces, ion interactions, hydrogen bonds, and/or hydrophobic interactions. The disclosed SLNs are water-insoluble complexes (e.g., ionic salt with hydrophobic counter-ions and covalent derivatives, such as esters or molecular associates, assembled by van der Waals' interactions), homogenized to submicron size using high-pressure homogenization.

SLNs built from waxes and/or glycerides have a high tendency for gelation during storage. Additionally, the solubility of many drugs in waxes and glycerides, particularly high-melting non-polar waxes and glycerides, is low. Initially-dissolved active components often separate from the lipid phase during storage, due to crystallization of either the lipid or the active components themselves. This is one of the main reasons for the physical instability of drug-loaded SLNs and nanoparticulate lipid conjugates (NLC) (Constantinides et al., Tocol emulsions for drug solubilization and parenteral delivery. Adv. Drug Deliv. Rev., 56(9):1243-1255, 2004).

Considering the limitations of conventional drug carriers, there exists a need to develop a biodegradable colloidal delivery system with an appropriate composition for the lipid phase, capable of controlled delivery of bioactive substances. Such a colloidal delivery system would overcome some or all of the drawbacks associated with traditional systems, including instability, toxicity, modification of biodistribution patterns, and manufacturing technology.

To increase solubility, more polar compounds may be explored (e.g., as monoglycerides or diglycerides) (Davis et al., Lipid emulsions as drug delivery systems. N Y Acad. Sci., 507:75-88, 1987). Free hydroxyl groups provide increased lipid phase polarity, resulting in improved solubility of polar compounds in the lipid phase. At the same time, though, monosubstituted or disubstituted glycerides tend to gelatinize in the presence of water, even at relatively low concentrations, causing aggregation and thereby rendering the suspension unsuitable for parenteral use (Massey, Interfacial properties of phosphatidylcholine bilayers containing vitamin E derivatives. Chem. Phys. Lipids, 109(2):157-174, 2001). Use of other organic materials (e.g., aromatic esters, cholesteryl derivatives, hydrophobic polymers, and the like) as major excipients for the lipid phase is strictly limited due to toxicity.

Tocopherol (or tocol) is a fat-soluble vitamin that is essential for normal reproduction, and is an important antioxidant that neutralizes free radicals in the body; it is also known as vitamin E. Tocopherol has been used in colloidal drug delivery systems, particularly in connection with emulsions, liposomes, lipospheres, and solid lipidic nanospheres, as either a therapeutic substance for delivery or a composition in the lipid phase of a drug delivery vehicle.

For example, U.S. Pat. No. 6,667,048 to Lambert et al. (“Emulsion vehicle for poorly soluble drugs”) describes the use of alpha-tocopherol, emulsified with tocopherol polyethylene glycol succinate (TPGS) and other non-ionic surfactants, in the preparation of a pharmaceutical emulsion vehicle with increased drug solubility and improved loading capacity. A combination of alpha-tocopherol and TPGS resulted in a stable emulsion capable of containing paclitaxel, etoposide, ibuprofen, griseofulvin, or vitamin E succinate, with concentrations of 1-10% in the lipid phase, or up to 2% in the final formulation. Similar compositions are disclosed in U.S. Pat. No. 6,193,985 to Sonne (“Tocopherol compositions for delivery of biologically active agents”), which describes use of tocopherol as a solvent and/or emulsifier for delivery of biologically active agents.

U.S. Pat. No. 6,479,540 to Constantinides et al. (“Compositions of tocol-soluble therapeutics”) describes compositions of tocol-soluble ion-pairs of biologically active components in liquid tocopherol. Alpha-D-tocopherol was used as a solvent; the ion-pairs were prepared separately, and the salt thus obtained was dissolved in the lipid phase, followed by subsequent emulsification. The ion-pair excipients which were investigated included different derivatives of tocopherols, phospholipids, and sterols, such as phosphates, succinates, sulfates, aspartates, and glutamates.

U.S. Pat. No. 6,193,985 to Sonne (“Tocopherol compositions for delivery of biologically active agents”) describes the use of a tocopherol, or a derivative thereof, as a solvent and/or emulsifier for substantially insoluble and sparingly soluble biologically active agents. The tocopherol composition is emulsified with non-ionic surfactant tocopherol polyethylene glycol succinate (TPGS), to form a drug-loaded emulsion capable of enhanced transmucosal delivery of biologically active agents.

U.S. Pat. No. 4,861,580 (“Composition using salt form of organic acid derivative of alpha-tocopheral”); U.S. Pat. No. 5,041,278 (“Alpha tocopherol-based vesicles”); U.S. Pat. No. 5,234,634 (“Method for preparing alpha-tocopherol vesicles”); and U.S. Pat. No. 5,364,631 (“Tocopherol-based pharmaceutical systems”), all to Janoff et al. describe the formation of liposomes comprising tocopherol hemisuccinate and/or cholesterol hemisuccinate salts of different amine-containing drugs. Salts of the hemisuccinates with tris(hydroxymethyl)aminomethane demonstrated detergent properties, and may be used for solubilization of hydrophobic drugs, such as pregnanolone, miconazole, or cyclosporin A. To prepare the liposomes, amine salts of the hemisuccinates (tris or pilocarpine salt) were dissolved in organic solvent; after solvent evaporation, the resulting film was hydrated, and then passed several times through membrane filters, in order to form multilamellar vesicles. Addition of tocopherol to the lipid phase increased viscosity of the liposomal preparations. As further disclosed by Massey (Interfacial properties of phosphatidylcholine bilayers containing vitamin E derivatives. Chem. Phys. Lipids, 109(2):157-174, 2001), the incorporation of different tocopheryl esters into the phospholipid bilayers of model membranes may change bilayer mobility, surface charge, and hydration.

Finally, U.S. Pat. No. 6,685,960 to Gasco (“Solid lipidic nanospheres suitable to a fast internalization into cells”) describes solid lipidic nanospheres comprising, as an active substance, a cytotoxic hydrophobic ester (e.g., butyrates of cholesterol, tocopherol, or glycerol), releasing butyric acid intracellularly, for use in treating tumors.

### SUMMARY OF THE INVENTION

The inventors have developed a solid lipid nanoparticle (SLN), comprising tocopherol or a derivative thereof or an obvious chemical equivalent thereof, for use in drug delivery. The inventors have also developed a colloidal drug delivery system comprising SLNs of the invention.

Accordingly, in one aspect, the present invention provides a drug carrier that includes a solid lipid nanoparticle, wherein the SLN includes tocopherol or a derivative thereof. In one embodiment, the tocopherol derivative is a tocopherol ester (e.g., tocopheryl palmitate, tocopheryl stearate, tocopheryl behenate, tocopheryl succinate, tocopheryl phosphate, tocopheryl enantate, tocopheryl acetate, or tocopheryl nicotinate). Also provided is a method for preparing the drug carrier. In one embodiment, the method does not use high-pressure homogenization. In another embodiment, the method does not use an organic solvent.

The SLN of the invention may be loaded with a water-insoluble or water-soluble biologically active compound. In one embodiment, the biologically active compound is water soluble. By way of example, and not of limitation, the water-soluble biologically active compound may be an antibiotic. By way of further example, the antibiotic may be selected from the group consisting of an aminoglycoside, a macrolide, a polypeptide, a fluoroquinolone, a penicillin, and a cephalosporin. Exemplary antibiotics include, without limitation, streptomycin, gentamicin, kanamycin, amikacin, neomycin, rifampicin, erythromycin, lincomycin, vancomycin, capreomycin, colistin, polymixin, gramicidin, ampicillin, cephalosporin, levofloxacin, moxifloxacin, and gatifloxacin.

The drug carrier of the present invention may further include a hydrophobic adjuvant. In one embodiment, the hydrophobic adjuvant is a charged compound. In another embodiment, the drug carrier is loaded with a water-soluble biologically active compound, and the hydrophobic adjuvant and the water-soluble biologically active compound have charged moieties of opposite signs.

In another aspect, the present invention provides a pharmaceutical composition that includes a solid lipid nanoparticle and a biologically active compound, wherein the SLN includes tocopherol or a derivative thereof. In one embodiment, the biologically active compound is water soluble (e.g., an antibiotic).

In yet another aspect, the present invention provides a colloidal drug delivery system comprising solid lipid nanoparticles (SLNs), wherein the SLNs comprise tocopherol or a derivative thereof. In one embodiment, the tocopherol derivative is a tocopherol ester (e.g., tocopheryl palmitate, tocopheryl stearate, tocopheryl behenate, tocopheryl succinate, tocopheryl phosphate, tocopheryl enantate, tocopheryl acetate, or tocopheryl nicotinate). Also provided is a method of preparing the colloidal drug delivery system. In one embodiment, the method does not use high-pressure homogenization. In another embodiment, the method does not use an organic solvent.

In the colloidal drug delivery system of the invention, at least some of the SLNs may be loaded with a biologically active compound. In one embodiment, the biologically active compound is water soluble. By way of example, and not of limitation, the water-soluble biologically active compound may be an antibiotic. By way of further example, the antibiotic may be selected from the group consisting of an aminoglycoside, a macrolide, a polypeptide, a fluoroquinolone, a penicillin, and a cephalosporin. Exemplary antibiotics include, without limitation, streptomycin, gentamicin, kanamycin, amikacin, neomycin, rifampicin, erythromycin, lincomycin, vancomycin, capreomycin, colistin, polymixin, gramicidin, ampicillin, cephalosporin, levofloxacin, moxifloxacin, and gatifloxacin.

In one embodiment of the present invention, the colloidal drug delivery system has a lipid phase, and at least 50% of the biologically active compound is associated with the lipid phase. In another embodiment, the colloidal drug delivery system is capable of controlled delivery of the biologically active compound. By way of example, and not of limitation, delivery may be effected via a parenteral, oral, nasal, pulmonary, rectal, topical, transdermal, or transmucosal route of administration.

The colloidal drug delivery system of the invention may further include a hydrophobic adjuvant. In one embodiment, the hydrophobic adjuvant is a charged compound. In another embodiment, at least some of the SLNs are loaded with a water-soluble biologically active compound, and the hydrophobic adjuvant and the water-soluble biologically active compound have charged moieties of opposite signs. The colloidal drug delivery system may also further include a stabilizer selected from the group consisting of an ionic or non-ionic surfactant and a phospholipid.

Other features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from reading the detailed description.

### DETAILED DESCRIPTION OF A PREFERRED EMBODIMENT

Solid lipid nanoparticles (SLNs) of submicron size, comprising lipid material that is solid at room temperature (i.e., the lipid material has a melting point no less than about 18° C.), have excellent potential as drug carriers, particularly in colloidal drug delivery systems, due to their perfect safety profiles (i.e., they are non-toxic, with components that are generally recognized as safe (GRAS)) and their biocompatibility, enzymatic degradability, and stability properties. The inventors have developed SLNs that include tocopherol or a derivative thereof (e.g., a tocopherol ester). The inventors have also developed a colloidal drug delivery system comprising the SLNs in combination with pharmaceutically-applicable excipients.

The SLNs of the invention are biodegradable, biocompatible, and non-toxic, and show improved chemical and physical stability during storage. The SLNs can be loaded with water-soluble and water-insoluble drugs, and can be prepared without the use of organic solvents and other potentially dangerous components. The colloidal system of the invention permits the controlled delivery of biologically active substances, such as drugs or other biological compounds, via parenteral, oral, nasal, pulmonary, rectal, topical, transdermal, transmucosal, and other routes of administration.

The Solid Lipid Nanoparticle (SLN)

The present invention provides a solid lipid nanoparticle (SLN) comprising tocopherol or a derivative thereof. As used herein, a “solid lipid nanoparticle”, or “SLN”, is a non-vesicular lipid aggregate, having a diameter of less than 1 micrometer (μm) (i.e., less than 1000 nm), that is solid at room temperature (i.e., having a melting point no less than about 18° C.). In one embodiment, the SLN of the invention has a diameter that is 10-990 nm (e.g., 100-450 nm). As further used herein, a “non-vesicular lipid aggregate” is a lipid structure which does not form a closed internal volume (vesicle); in particular, it is neither a unilamellar nor a multilamellar liposome.

As discussed above, tocopherol (or tocol) is a fat-soluble vitamin that is essential for normal reproduction, and is an important antioxidant that neutralizes free radicals in the body; it is also known as vitamin E. The tocopherol molecule comprises a relatively polar aromatic core and a more hydrophobic non-polar aliphatic tail; thus, solubilization properties for vitamin E are much higher than those for aliphatic glycerides, esters, and waxes (U.S. Pat. No. 6,479,540 to Constantinides et al. (“Compositions of tocol-soluble therapeutics”); U.S. Pat. No. 4,861,580 (“Composition using salt form of organic acid derivative of alpha-tocopheral”); U.S. Pat. No. 5,041,278 (“Alpha tocopherol-based vesicles”); U.S. Pat. No. 5,234,634 (“Method for preparing alpha-tocopherol vesicles”); and U.S. Pat. No. 5,364,631 (“Tocopherol-based pharmaceutical systems”), all to Janoff et al.). All tocopherol derivatives have low toxicity. In addition, esterification of the tocopherol core does not eradicate vitamin activity of the resulting esters. All tocopherol esters that are susceptible to hydrolysis (e.g., tocopheryl acetate, nicotinate, palmitate, stearate, erucate, behenate, phosphate, succinate, and the like) are a source of vitamin E.

The SLN of the present invention is prepared from material that is solid at room temperature (RT). Free tocopherol cannot be used, because it is liquid at RT. Accordingly, the SLN of the invention may comprise a solid tocopherol ester with an appropriate melting point in accordance with those noted above (e.g., from 24° C., for D-alpha-tocopherol acetate, to 76° C., for D-alpha-tocopherol succinate). In one embodiment of the present invention, the lipid of the SLN contains a tocopherol ester with a melting point of 20° C. or higher. In another embodiment, the tocopherol ester has a melting point greater than 21° C., 22° C., 23° C., 24° C., or 25° C. Exemplary tocopherol esters for use in the present invention include, without limitation, tocopheryl palmitate, tocopheryl stearate, tocopheryl behenate, tocopheryl succinate, tocopheryl phosphate, tocopheryl enantate, tocopheryl acetate, and tocopheryl nicotinate. The tocopherol esters may be used alone or in any desired combination. Exemplary solid esters of tocopherol (with associated melting point) include, without limitation, acetate (+24° C.), butyrate (+20° C.), palmitate (+33° C.), stearate (+36° C.), nicotinate (+42° C.), behenate (+45° C.), and succinate (+76° C.).

The bioavailability of biologically active compounds can be enhanced by incorporating the compounds into the SLNs of the invention, such that they are solubilized in the nanosized lipid matrices. Accordingly, the SLN of the invention may be loaded with a biologically active compound, for delivery to a subject or target. By way of example, the biologically active compound may include, without limitation, a biologically active antibiotic, protein, peptide, polysaccharide, or cardiovascular drug. In one embodiment, the biologically active compound is water soluble. As used herein, a “water-soluble biologically active compound” includes any biologically active compound with solubility in water that is high enough to provide a water solution suitable for demonstration of the compound's biological activity. For example, the water-soluble biologically active compound may be a pro-drug (a compound that is further processed to bioactive form) or an antibiotic.

In one embodiment, the invention relates to a particulate pharmaceutical composition comprising a water-soluble antibiotic associated with the solid lipid aggregates of submicron size, wherein the lipids are in a solid state at room temperature, and the lipid phase of submicron aggregate contains tocopheryl esters. By way of example, the antibiotic may be an aminoglycoside, macrolide, polypeptide, fluoroquinolone, penicillin, or cephalosporin. Exemplary antibiotics include, without limitation, streptomycin, gentamicin, kanamycin, amikacin, neomycin, rifampicin, erythromycin, lincomycin, vancomycin, capreomycin, colistin, polymixin, gramicidin, ampicillin, cephalosporin, levofloxacin, moxifloxacin, and gatifloxacin.

The solid lipid nanoparticle of the present invention may further comprise a hydrophobic adjuvant. As used herein, “hydrophobic adjuvant” means a hydrophobic compound that interacts with a biologically active compound incorporated into a nanoparticle, providing a complex with better solubility in a lipid core of the nanoparticle and/or better integration with the interface of the nanoparticle. In one embodiment, the adjuvant is a charged compound. In another embodiment, the adjuvant and the biologically active compound contain charged moieties of opposite signs. The solid lipid nanoparticle of the present invention may also further comprise a stabilizer (e.g., a stabilizer selected from the group of ionic or non-ionic surfactants or phospholipids).

Due to its solid nature, the lipid phase of the SLN described herein is more resistant to coalescence than liquid droplets in emulsions. The SLNs of the invention have improved physical stability, and can be lyophilized to reach more stable anhydrous systems. Lyophilized SLN powders of the invention can be reconstituted more easily than freeze-dried oil-in-water emulsions.

The lipophilic nature of the SLNs of the invention also makes them appropriate for the incorporation of lipophilic substances by solubilization in the lipid matrix. In one embodiment of the present invention, the biologically active compound is associated with the lipid phase of the SLN or the colloidal drug delivery vehicle comprising same. For example, at least 50% of the biologically active compound may be associated with the lipid phase of the SLN. Incorporation of water-soluble compounds into the SLNs can be improved by hydrophobization, using ion-pair formation or another type of modification known in the art.

The Colloidal Drug Delivery System

The present invention also provides a colloidal vehicle comprising SLNs of the invention. As used herein, a “colloidal drug delivery system”, or “colloidal vehicle”, is a system comprising a plurality of separate small particles of biocompatible material, finely dispersed in liquid media. In one embodiment of the invention, the colloidal-drug delivery system is loaded with at least one biologically active compound, and is designed for delivery of the incorporated material to a subject, in order to treat a disease or malfunction.

The colloidal drug delivery system of the invention may have a sustained release, physically stable, chemically stable, and biocompatible lipid phase, which is solid at room temperature. The solid lipid phase provides for sustained release of incorporated material (e.g., a drug), as compared with fluid emulsion droplets, due to restricted diffusion.

The lipid phase of the colloidal drug delivery system includes SLNs comprising tocopherol or a derivative thereof. Suitable tocopherol-based derivatives include those having appropriate melting points (e.g., higher than 18° C.). In one embodiment, the SLNs comprise a tocopherol that does not contain free non-esterified tocopherol, or at least does not contain such free non-esterified tocopherol in an amount which may decrease the melting point below the preferable range. The colloidal vehicle of the invention may further comprise a hydrophobic adjuvant. Additionally, the colloidal vehicle may further comprise a stabilizer (e.g., a stabilizer selected from the group of ionic or non-ionic surfactants or phospholipids).

Targeting of a colloidal system comprising a drug-loaded SLN can be regulated by modifying the SLN surface, changing the particle size, and/or changing the composition of the colloidal system's lipid phase or surfactants. By such modifications, the SLN of the system can acquire stealth properties, be masked from uptake by the reticulo-endothelial system (RES), and be targeted to macrophages, brain, lungs, liver, or other cells, tissues, or organs. In one embodiment, at least 50% of the biologically active compound may be associated with the lipid phase of the colloidal vehicle.

Compositions Comprising the SLNs

The present invention further provides a composition comprising a solid lipid nanoparticle of the invention, or a colloidal vehicle comprising same, and a pharmaceutically-acceptable carrier. Suitable pharmaceutically-acceptable carriers are known in the art, and are described, for example, in Remington's Pharmaceutical Sciences (Easton, Pa.: Mack Publishing Company, 1985) and in the Handbook of pharmaceutical Additives, compiled by Michael and Irene Ash (Aldershot, UK: Gower Publishing Limited, 1995). The composition of the invention can be lyophilized and reconstituted for administration to a patient in need thereof. In one aspect, the pharmaceutical composition of the invention can be used to enhance biodistribution and drug delivery of hydrophilic or water-soluble drugs.

The pharmaceutical composition of the invention may be administered to living subjects, including humans and animals, by any convenient route of administration known in the art. By way of example, the pharmaceutical composition may be administered by direct application to the infected site (e.g., by subcutaneous injection, by intravenous injection, or by other type of injection), or by oral, parenteral, peroral, nasal, pulmonary, rectal, topical, transdermal, or transmucosal administration. In the case of respiratory infections, it may be desirable to administer the colloidal vehicles or solid lipid nanoparticles of the invention, and compositions comprising same, through techniques known in the art. Depending upon the route of administration (e.g., injection, topical, oral, inhalation, or other administration route), the pharmaceutical composition, colloidal vehicle, solid lipid nanoparticle, or drug of the invention may be coated in a material that will protect it from the action of enzymes, acids, and other natural conditions that may inactivate the ingredients and components contained therein.

The compositions described herein can be prepared by methods known in the art for the preparation of pharmaceutically-acceptable compositions. Furthermore, the compositions can be administered to subjects such that an effective quantity of the active substance (e.g., a hydrophobic drug, such as cyclosporin) is combined in a mixture with a pharmaceutically-acceptable carrier. The compositions may include, without limitation, solutions of the substances in association with one or more pharmaceutically-acceptable vehicles or diluents; moreover, they may be contained in buffered solutions with a suitable pH, and/or they may be iso-osmotic with physiological fluids.

In addition to pharmaceutical compositions, compositions for non-pharmaceutical purposes are also included within the scope of the present invention. For example, compositions for non-pharmaceutical use may include diagnostic or research tools. In one embodiment, the drug, or a colloidal vehicle or solid lipid nanoparticle comprising the drug, can be labeled with a label known in the art (e.g., a florescent label, a radio label, etc.).

Method of Manufacturing the SLNs and Colloidal Vehicles

The SLNs of the present invention, and colloidal vehicles comprising same, may be prepared for parenteral, oral, nasal, pulmonary, rectal, topical, transdermal, transmucosal, or other administration by a simplified method that does not use toxic organic solvents or high-pressure homogenization (i.e., high forces and high energy are not applied, thereby allowing the incorporation of sensitive and unstable compounds). The SLNs and colloidal vehicles prepared by this method lack many of the problems associated with conventional colloidal delivery systems.

By way of example, and not of limitation, the method for preparing the SLNs of the present invention, or a colloidal vehicle comprising same, may comprise the steps of: (a) combining and melting lipid components, surfactants, and other additives, to make a liquefied mixture; (b) adding at least one biologically active component (e.g., an antibiotic) to the liquefied mixture; (c) adding an aqueous phase (e.g., hot water, in whole or in part), and intensively mixing the resulting preparation; and (d) filtering the preparation to eliminate undissolved particles. In one embodiment, the method of the present invention does not use organic solvents. In another embodiment, the method does not use high-pressure homogenization. In still another embodiment, the SLNs or vehicles of the invention may be loaded with at least one biologically active compound for medicinal use.

In accordance with the method of the present invention, a conjugate linking the biologically active compound and an associated modifying molecule may be prepared prior to, or at the same time as, the SLNs of the invention are prepared. The SLNs may also further comprise a hydrophobic adjuvant, wherein the biologically active compound interacts with the charged hydrophobic adjuvant “in situ” during the lipid aggregate formation.

Applications for the SLNs and Colloidal Vehicles

In one embodiment, the solid lipid nanoparticle or colloidal vehicle of the invention is useful as a medicinal preparation for administration to a patient in need thereof. In another embodiment, the vehicle or SLN is useful in the preparation of a medicament comprising a prophylactic or therapeutic compound. In yet another embodiment, the vehicle or SLN is useful in the delivery of a compound to a patient in need thereof. In still another embodiment, the colloidal vehicle or SLN of the invention is loaded with an antibiotic, and is useful in the preparation of a medicament for treatment of a bacterial infection.

The present invention is described in the following Examples, which are set forth to aid in the understanding of the invention, and should not be construed to limit in any way the scope of the invention as defined in the claims which follow thereafter.

### EXAMPLES

### Example 1

Preparation of Palmitic Ester of Tocopherol

43.08 g (0.1 mole) of (±)-DL-tocopherol (99% purity) was dissolved in 200 ml of anhydrous tetrahydrofuran (THF). The solution was cooled with ice, and 10.12 g (0.1 mole) of triethylamine (99.5% purity; d=0.726) was added. This step was followed by the addition of a solution of 27.5 g (0.1 mole) of palmitoyl chloride (purity 97.9%; d=0.907) in 100 ml of THF while stirring. The reaction was carried out at room temperature for 4 hours, heated to boiling for 2 hours, and controlled by thin layer chromatography. After completion, THF was evaporated, and the solidified product was crystallized from ethyl alcohol. The yield was 91%, with a melting point (uncorr.) of +33° C.

Tocopheryl stearate and other esters may be prepared in a similar manner.

### Example 2

Streptomycin-Loaded Solid Lipid Colloidal Delivery System

Solid lipid nanoparticles with streptomycin were prepared using a mixture of tocopheryl palmitate and tocopheryl succinate esters.

TABLE 1
Streptomycin-loaded solid lipid nanoparticles (tocopherol esters)

| | Ex. 2 |
|---|---|
| Component | Weight, mg |
| LIPID PHASE | |
| Tocopheryl palmitate | 300 |
| Tocopheryl succinate | 700 |
| Streptomycin sulfate (potency 650 μg/mg) | 70 |
| Cholesteryl sulfate potassium | 30 |
| Lecithin (Phospholipon ® S-80)* | 150 |
| Tyloxapol ™ | 250 |
| Cremophor ® EL | 350 |
| AQUEOUS PHASE | |
| Sodium citrate anhydrous | 230 |
| Water purified (70° C.) | to 20 ml |

*lecithin was used as a 50% solution

All components of the lipid phase were combined, heated to 45-55° C., and mixed until an homogenous mixture was obtained. The water phase was heated to 60-70° C., and added to the lipid phase with intensive stirring (2,000-5,000 rpm) using an appropriate rotor-stator mixer. Mixing was continued for 5 minutes, and then the suspension was filtered through a 0.45-μm nylon membrane filter (25-mm syringe filter; Pall) to separate possible metal particles and aggregates.

It was found that the more polar succinate ester was located on the superficial interface of the nanoparticles. At pH 5.5-6.5, obtained by buffering with sodium citrate, the tocopheryl succinate was partially ionized and negatively charged, thereby providing electrostatic stabilization due to repulsion. Cholesteryl sulfate was used as a counter-ion for improving of streptomycin entrapment. Lecithin (final concentration 0.75%) was used as a co-surfactant and stabilizer of the formed suspension. Part of the succinate ester can also be introduced in the phospholipid bilayer, to improve stability. Absence of vesicular structures was confirmed by centrifugation of the resulting suspension at 12,000 g for 15 minutes; no pellet was formed.

The particle size of the resulting suspension-was determined by laser diffraction using a laser diffraction particle size analyzer SALD 2001 (Shimadzu, Japan). 90% of the particles had a diameter (D90) below 386 nm; the median diameter (D50) was 131 nm. The preparation was stable at room temperature.

### Examples 3-4

Examples 3 and 4 show preparation of mixed micellar solid lipid aggregates. These formulations contain no non-ionizable lipid, and differ only by the type of phospholipid used and by the use of hydrogenated or non-hydrogenated soy lecithin.

TABLE 2
Streptomycin-loaded micellar solid lipid aggregates

| | Ex. 3 | Ex. 4 |
|---|---|---|
| Component | Weight, g | |
| LIPID PHASE | | |
| Tocopherol succinate | 1.8 | 1.8 |
| Tyloxapol ™ | 1.1 | 1.0 |
| Streptomycin sulfate (potency 650 μg/mg) | 0.25 | 0.25 |
| Cholesteryl sulphate (potassium salt) | 0.13 | 0.13 |
| Lecithin (Phospholipon ® S-80) 50% solution | 2.0 | |
| Hydrogenated lecithin (Phospholipon ® H-80), | | 2.0 |
| 50% suspension | | |
| AQUEOUS PHASE | | |
| Sodium citrate anhydrous | 0.92 | 0.92 |
| Water purified (70° C.) | 43.8 | 43.9 |
| Total weight, g | 50.0 | 50.0 |

| | |
|---|---|
| Appearance after 1 month of storage at RT | stable suspensions |

Absence of vesicular structures was confirmed by centrifugation of the resulting suspensions at 12,000 g for 15 minutes. The resulting colloidal formulations, according to observed physical properties, comprised mixed micelles comprising surfactant, drug associated with a counter-ion, a tocopherol ester, and phospholipids, evenly distributed in the water phase. All components of the lipid phase were solid; thus, the formed solid lipid aggregates provided marked retention and release of the included drug, due to the high intrinsic viscosity and correspondingly high diffusion in these lipid nanoparticles.

### Examples 5-10

Examples 5-10 demonstrate the influence of different components on properties of the prepared formulations.

TABLE 3
Streptomycin-loaded solid lipid nanoparticles

| Component | Ex. 5 | Ex. 6 | Ex. 7 | Ex. 8 | Ex. 9 | Ex. 10 |
|---|---|---|---|---|---|---|
| LIPID PHASE | | | | | | |
| Tocopherol palmitate | | 1.0 | 1.0 | | | |
| Tocopherol acetate USP | | | | 1.0 | 1.3 | |
| (Covitol ® 1360, Henkel) | | | | | | |
| Tocopherol stearate | | | | | | 1.0 |
| Tocopherol succinate | 1.8 | 0.8 | 0.8 | 0.8 | 1.2 | 1.2 |
| Suppocire ® CM (Gattefosse) | 4.5 | | | | | |
| Syncrowax ® (Croda) | | 3.5 | 3.5 | | | |
| Glycerol tribehenate | | | | 3.5 | 4.0 | 3.5 |
| (Pelemol ™, Phoenix | | | | | | |
| Chemicals) | | | | | | |
| Streptomycin sulfate | 0.125 | 0.5 | 0.5 | 0.5 | 0.5 | 0.5 |
| (potency 650 μg/mg) | | | | | | |
| Cholesteryl sulfate K | | 0.13 | 0.13 | 0.13 | | |
| Cetyl phosphate | 0.10 | | | | | |
| (Hostaphat ™ CC100, | | | | | | |
| Clariant) | | | | | | |
| Stearic acid | | 0.06 | 0.06 | 0.06 | 0.06 | |
| Tocophersolan ® (Eastman) | | 1.0 | 1.0 | 1.0 | 1.0 | 1.0 |
| Tyloxapol ™ (Aldrich) | 1.1 | | | | | |
| Cremophor ® EL (BASF) | 1.1 | 1.0 | | 1.0 | 1.0 | 1.0 |
| Pluronic F-68 | | | 1.0 | | | |
| 50% Lecithin solution | 2.0 | 0.6 | 0.6 | 0.6 | 0.6 | 0.6 |
| (Phospholipon S-80) | | | | | | |
| AQUEOUS PHASE | | | | | | |
| Sodium citrate (dihydrate) | 0.45 | 0.5 | 0.5 | 0.5 | 0.5 | 0.5 |
| Water 60° C. | 60 | 80 | 80 | 80 | 80 | 80 |
| Median diameter, nm (D50) | 330 | 190 | 540 | 255 | 280 | 300 |
| Drug inclusion, % | 61 | 58 | 55 | 66 | 52 | 59 |
| Appearance after 2 months at | stable | stable | separation | stable | separation | stable |
| RT | | | | | | |

The preparation process for Examples 5-10 was similar to that for Examples 2-4, except that Examples 7-9 were also treated with high-pressure homogenization (Emulsiflex C-5, Avestin, Ottawa), at 12,000 psi, for 5 cycles. The resulting suspensions were centrifuged at 3,000 g for 20 minutes, and filtered through 0.45 μm of nylon membrane.

The additional application of high-pressure homogenization does not necessarily improve stability of the formulation. The formulations were not very sensitive to the chemical structure of counter-ions: the appropriate levels of different counter-ions (e.g., aliphatic stearic acid, cetylphosphate, aromatic cholesteryl sulfate, and tocopheryl phosphate) provided stable submicron suspensions with a good level of drug association with the lipid particles. Nevertheless, the absence of a strong counter-ion may result in an unstable product (Example 9).

Since the prepared formulations had a high ratio of lipid:phospholipids, with a relatively high level of surfactants and a low final concentration of phospholipids (1-2% of the total), the formation of vesicles (e.g., liposomes) was hardly feasible. Absence of vesicular structures was confirmed by centrifugation of the resulting suspensions at 12,000 g for 15 minutes; no pellet was formed. In the resulting formulations, tocopherol ester and triglyceride formed a lipid core of the nanoparticle. This core was surrounded with a layer of tocopherol succinate, phospholipid, and surfactant on the interface. Drug associated with the counter-ion (phosphate, sulfate, or succinate) formed an “in situ” soluble aminoglycoside drug; the low-solubility counter-ion formed an insoluble salt during preparation.

The degree of antibiotic association with the colloidal delivery system (drug inclusion) was evaluated using Ultrafree™-MC ultrafiltration centrifuge device (Millipore) with a cellulose membrane (cutoff 30,000 dalton) at 10,000 rpm. Drug content in analytes was determined using an HPLC method.

Gentamicin-Loaded Solid Lipid Colloidal Delivery System

Another aminoglycoside antibiotic, gentamicin, was introduced into the lipid colloidal delivery system using a similar approach. Gentamicin formulations are more sensitive to composition and process variables. Nevertheless, formulations (Examples 18-19) that were prepared showed a good level of drug inclusion and reasonable physical and chemical stability.

### Examples 11-19

TABLE 4
Gentamicin-loaded micellar solid lipid aggregates

| | Ex. | Ex. | Ex. | Ex. | Ex. | Ex. | Ex. | Ex. | Ex. |
|---|---|---|---|---|---|---|---|---|---|
| Components | 11 | 12 | 13 | 14 | 15 | 16 | 17 | 18 | 19 |
| LIPID PHASE | | | | | | | | | |
| Tocopherol succinate | 1.0 | 1.0 | 0.67 | 0.7 | 1.8 | 4.0 | 2.0 | 2.4 | 2 |
| Tyloxapol ™ (Aldrich) | 0.32 | 0.65 | 0.63 | 0.75 | 1.8 | 2.0 | 0.47 | 1.36 | 1.23 |
| Cremophor ® EL | | | | | | | 0.80 | 1.0 | |
| (BASF) | | | | | | | | | |
| Gentamicin sulfate | 0.60 | 0.68 | 0.08 | 0.12 | 0.20 | 0.40 | 0.26 | 0.32 | 0.27 |
| Cholesteryl sulfate | | 0.62 | | 0.1 | 0.18 | 0.27 | 0.18 | 0.22 | 0.185 |
| potassium salt | | | | | | | | | |
| Cetylphosphate | 0.35 | | 0.07 | | | | | | |
| (Hostaphat ™ CC100, | | | | | | | | | |
| Clariant) | | | | | | | | | |
| Sodium deoxycholate | | | 0.04 | | | | | | |
| 50% Lecithin solution | 0.8 | 1.5 | 0.5 | 0.75 | 1.65 | 2.5 | 1.5 | 2.78 | 2.1 |
| (Phospholipon S-80) | | | | | | | | | |
| AQUEOUS PHASE | | | | | | | | | |
| Sodium citrate | 0.04 | 0.04 | 0.04 | 0.040 | 0.40 | 0.40 | 0.12 | 0.16 | 0.47 |
| (anhydrous) | | | | | | | | | |
| Hot water (>70° C.), ml | 4 | 4 | 4 | 4 | 6 | 20 | 10 | 10 | 10 |
| Cold water, ml | to 20 | to 20 | to 20 | to 20 | to 60 | to | to 50 | to 50 | to 50 |
| | | | | | | 100 | | | |
| Filtration via 0.45-μm | −− | −− | −− | ++ | ++ | ± | −− | ++ | ++ |
| filter | | | | | | | | | |
| Median diameter, nm | n/a | 102 | 360 | 111 | 176 | 120 | 155 | 90 | 122 |
| (D50) | | | | | | | | | |
| Drug inclusion, % | | | | | | | | 58 | 55 |
| Appearance after 2 | gel | gel | separation | gel | separation | gel | gel | stable | stable |
| months at RT | | | | | | | | | |

### Examples 11-19

Tocopherol succinate, Tyloxapol, Cremophor, and Cetylphosphate or Cholesteryl sulfate were melted together using a water bath (75-80° C.). To the melted mixture were added lecithin and a dry powder of gentamicin sulfate; the components were mixed at 65-70° C. for 5 minutes. 10-20% of the total amount of the water phase, heated to 70-80° C., was added to the lipid-surfactant mixture; this was then mixed for 5 minutes. After formation of an homogeneous mixture, the remaining amount of the water phase was added, and the mixture was mixed for 5 minutes (at 2,000-5,000 rpm) using an appropriate rotor-stator mixer. The resulting suspension was filtered through a 0.45-μm membrane filter (25-mm nylon syringe filter; Pall), to separate possible metal particles and aggregates.

### Examples 20-26

TABLE 5
Gentamicin-loaded solid lipid nanoparticles

| SLN | Ex. 20 | Ex. 21 | Ex. 22 | Ex. 23 | Ex. 24 | Ex. 25 |
|---|---|---|---|---|---|---|
| LIPID PHASE | | | | | | |
| Tocopherol succinate | 2.0 | 2.0 | 2.0 | 0.6 | 0.4 | 0.6 |
| Tocopherol nicotinate | | 1.0 | | | | |
| Tocopherol phosphate | | | | | 0.25 | 0.033 |
| disodium salt | | | | | | |
| Tocopheryl palmitate | | | | | 0.5 | |
| Suppocire ® CM | | 2.0 | 4.0 | 1.5 | | 1.5 |
| Syncrowax HDC (glyceryl | | | | | 1.8 | |
| tribehenate, Croda) | | | | | | |
| Gentamicin sulfate | | | 0.5 | 0.041 | 0.50 | 0.045 |
| Gentamicin cetylphosphate | 0.80 | 0.80 | | | | |
| salt | | | | | | |
| Cholesteryl sulfate | | | 0.34 | | | |
| potassium salt | | | | | | |
| Cetylphosphate | | | | 0.031 | | |
| (Hostaphat ™ CC100, | | | | | | |
| Clariant) | | | | | | |
| Tocophersolan ® (Eastman) | | 1 | 1.0 | | 0.5 | |
| Tyloxapol ™ (Aldrich) | 0.8 | | | 0.34 | | 0.32 |
| Cremophor ® EL (BASF) | 1 | 1 | 1.25 | 0.4 | 0.5 | 0.4 |
| 50% Lecithin solution | 1.14 | 1 | 1.0 | 0.65 | 0.30 | 0.65 |
| (Phospholipon S-80) | | | | | | |
| AQUEOUS PHASE | | | | | | |
| Sodium citrate (dihydrate) | | 0.1 | 0.25 | 0.06 | 0.25 | 0.16 |
| Water (>70° C.) | 44.3 | 41.1 | 45 | 4 | 35 | 4 |
| Cold water | | | | 16 | | 16 |
| Final volume, ml | 50 | 50 | 50 | 20 | 40 | 20 |
| Filtration via 0.2-μm filter | ± | ± | ++ | +++ | + | +++ |
| Appearance after 2 months at | separation | separation | stable | stable | viscous | stable |
| RT | | | | | | |

Gentamicin-loaded lipid nanoparticles were prepared in a manner similar to that used for the streptomycin SLNs. For Examples 20-21, cetylphosphate salt of gentamycin was prepared separately by mixing an alcoholic solution of cetylphosphate and an aqueous solution of gentamicin sulfate in equimolar ratio. The precipitated salt was separated, washed with purified water, and dried at 40° C. The formulations of Examples 24-25 were also homogenized using a high-pressure homogenizer (Avestin Emulsiflex® C-5), at 15,000 psi, for 5 cycles. All samples were centrifuged and filtrated through a 0.45-μm membrane filter.

### Examples 26-28

Solid lipid nanoparticles with amikacin sulfate, neomycin sulfate, and kanamycin sulfate were prepared in a manner similar to that used in Example 19, with a final concentration of approximately 5 mg/ml.

TABLE 6
Solid lipid nanoparticles loaded with amikacin, neomycin, and kanamycin

| Components | Ex. 26 | Ex. 27 | Ex. 28 |
|---|---|---|---|
| LIPID PHASE | | | |
| Tocopherol succinate | 2 | 2 | 2 |
| Tyloxapol ™ (Aldrich) | 1.25 | 1.25 | 1.25 |
| Cremophor ® EL (BASF) | | | |
| Amikacin sulfate | 0.25 | | |
| Neomycin sulfate | | 0.25 | |
| Kanamycin sulfate | | | 0.25 |
| Cholesteryl sulfate potassium salt | 0.18 | 0.18 | 0.18 |
| 50% Lecithin solution | 2.0 | 2.0 | 2.0 |
| (Phospholipon S-80) | | | |
| AQUEOUS PHASE | | | |
| Sodium citrate (anhydrous) | 0.5 | 0.5 | 0.5 |
| Hot water (>70° C.), ml | 10 | 10 | 10 |
| Cold water, ml | 43.8 | 43.8 | 43.8 |
| Filtration via 0.2-μm filter | ++ | ++ | ++ |
| Appearance after 2 months at RT | stable | stable | stable |

Rifampicin, a potent antibiotic with pronounced antituberculosic activity, was successfully incorporated into the solid lipid colloidal delivery system. Since rifampicin is more hydrophobic than aminoglycosides, its incorporation may reach 98-99%.

### Examples 29-33

TABLE 7
Rifampicin-loaded micellar solid lipid aggregates

| Components | Ex. 29 | Ex. 30 | Ex. 31 | Ex. 32 | Ex. 33 |
|---|---|---|---|---|---|
| LIPID PHASE | | | | | |
| Tocopherol succinate | 0.8 | 4.0 | 6 | 3 | 4 |
| Tyloxapol ® (Aldrich) | 0.33 | | 1.5 | 1 | |
| Tocophersolan ® (Eastman) | | 1.5 | 0.5 | − | 1.5 |
| Rifampicin | 0.38 | 2.0 | 2.5 | 1.0 | 2.0 |
| Cholesteryl sulphate potassium salt | 0.160 | 0.8 | 1.27 | 0.63 | 1.27 |
| EtOH (g) | | | | − | |
| 50% Lecithin solution (Phospholipon S-80) | | 4.0 | 4.0 | 1.85 | 4.0 |
| Hydrogenated lecithin (Phospholipon S-80H), | 1.0 | | | | |
| 50% suspension | | | | | |
| AQUEOUS PHASE | | | | | |
| Sodium citrate (anhydrous) | | 0.12 | 0.4 | 0.2 | 0.4 |
| Arginine (base) | 0.12 | 0.3 | 0.2 | | |
| Hot water (>70° C.) | 4 | 20.0 | 20 | | 20 |
| Water | to 20 ml | to 100 ml | to 100 ml | to 40 ml | to 100 ml |
| Median diameter, nm (D50) | 170 | 68 | 123 | 101 | 154 |
| Drug inclusion, % (ultrafiltration) | | 98 | | | 92 |
| Filtration via 0.2-μm filter | ++ | +++ | ± | + | +++ |
| Appearance after 2 months at RT | viscous | stable | gel | separation | stable |

A formulation of rifampicin in solid lipid aggregates was prepared by a method similar to that used in Examples 11-19. Components of the lipid phase were mixed together, heated to 65-75° C. using a water bath while stirring for 15-20 minutes; hot water was then added. The resulting suspension was mixed for 5 minutes at 60-70° C. Formulations of Examples 30-32 were also treated using a high-pressure homogenizer (Avestin Emulsiflex® C-5), at 15,000 psi, for 5 cycles. All samples were centrifuged and filtrated through a 0.45-μm membrane filter. According to observed results, additional homogenization does not necessarily improve suspension stability.

### Examples 34-43

TABLE 8
Rifampicin-loaded solid lipid nanoparticles

| | | | | | Ex. | Ex. | | | Ex. | |
|---|---|---|---|---|---|---|---|---|---|---|
| Components | Ex. 34 | Ex. 35 | Ex. 36 | Ex. 37 | 38 | 39 | Ex. 40 | Ex. 41 | 42 | Ex. 43 |
| LIPID PHASE | | | | | | | | | | |
| Tocopherol succinate | 1.0 | 1.5 | 1.0 | 2.00 | 2.50 | 1.00 | 1.00 | 1.50 | 2.50 | 0.83 |
| Tocopherol | | | | 1.00 | | | | | | |
| nicotinate | | | | | | | | | | |
| Suppocire ® CM | 6.0 | 8.0 | 6.0 | 1.00 | 5.00 | | | | | |
| Syncrowax ® | | | | | | 6.00 | 5.00 | 5.00 | 5.00 | 5.00 |
| Stearic acid | | | | | | | | | | 0.09 |
| Cholesteryl oleate | | | | 1.00 | | | | | | |
| Rifampicin | 0.5 | 1 | 0.50 | 1.00 | 1.03 | 2.00 | 1.00 | 1.00 | 0.75 | 0.50 |
| Cholesteryl sulphate | | | 0.25 | 0.50 | | | | 0.48 | 0.35 | 0.25 |
| potassium salt | | | | | | | | | | |
| Cetylphosphate | 0.7 | 0.9 | | 0.46 | 0.43 | 0.83 | 0.43 | | | |
| (Hostaphat ™ | | | | | | | | | | |
| CC100) | | | | | | | | | | |
| Tocophersolan ® | 0.8 | 0.8 | 0.8 | 0.92 | 1.00 | 1.42 | 1.28 | 1.00 | 1.00 | 1.0 |
| (Eastman) | | | | | | | | | | |
| Cremophor ® EL | 0.8 | 1.0 | 1.0 | | | 0.90 | | 1.00 | | 1.0 |
| (BASF) | | | | | | | | | | |
| Tyloxapol ® | | | | 1.0 | 1.0 | | 1.0 | | 1.00 | |
| (Aldrich) | | | | | | | | | | |
| Ethanol 95% USP | | | 1.4 | | | | | | | |
| 50% Lecithin | 1.0 | 1.0 | 1.0 | 1.00 | 1.54 | 1.00 | 1.00 | 1.15 | 1.40 | 0.50 |
| solution | | | | | | | | | | |
| (Phospholipon S-80) | | | | | | | | | | |
| AQUEOUS | | | | | | | | | | |
| PHASE | | | | | | | | | | |
| Sodium citrate | | | | 0.12 | 0.50 | 0.50 | 0.62 | 0.53 | 0.50 | 0.50 |
| (dihydrate) | | | | | | | | | | |
| Arginine (base) | | | | | | | | 0.30 | | |
| Water 60° C. | 39.2 | 35.8 | 38.1 | 40.0 | 37.0 | 36.35 | 63.7 | 38.0 | 87.5 | 90.2 |
| Median diameter, nm | 50 | 50 | 50 | 50 | 50 | 50 | 75 | 50 | 100 | 100 |
| (D50) | | | | | | | | | | |
| Filtration via 0.2-μm | ± | ± | − | + | ++ | ++ | +++ | +++ | +++ | +++ |
| filter | | | | | | | | | | |
| Appearance after 2 | separation | precipitate | precipitate | separation | stable | gel | stable | stable | stable | stable |
| months at RT | | | | | | | | | | |

Preparation of rifampicin formulations in solid lipid nanoparticles was carried out by a method similar to that used in Examples 20-25. The components of the lipid phase were mixed together, and heated to 65-75° C. using a water bath, while stirring, for 15-20 minutes; hot water was then added. The resulting suspension was mixed for 5 minutes at 60-70° C. The formulations of Examples 36-37 were also treated using a high-pressure homogenizer (Avestin Emulsiflex® C-5), at 15,000 psi, for 5 cycles. All samples were centrifuged, and then filtered that a 0.45-μm membrane filter.

Addition of sodium citrate and/or arginine base to the colloidal lipid suspensions regulated their stability, and was sensitive to the pH of the compositions. Optimal stability (either physical stability of the suspension or chemical stability of rifampicin) was observed in the pH range from about 5.5 to about 7.5.

Polymixin is a basic polypeptide-type antibiotic. Inclusion of polymixin in the lipid colloidal delivery system may decrease nephrotoxicity of the drug and improve biodistribution.

### Examples 44-51

TABLE 9
Polymixin-loaded solid lipid nanoparticles and aggregates

| Components | Ex. 44 | Ex. 45 | Ex. 46 | Ex. 47 | Ex. 48 | Ex. 49 | Ex. 50 | Ex. 51 |
|---|---|---|---|---|---|---|---|---|
| LIPID PHASE | | | | | | | | |
| Tocopherol succinate | 0.56 | 0.63 | 0.60 | 0.70 | 0.55 | 1.25 | 1.20 | 0.6 |
| Suppocire ® CM | 1.00 | 1.00 | 1.20 | 1.20 | 1.20 | 2.00 | 2.00 | — |
| Polymixin sulfate | 0.07 | 0.08 | 0.09 | 0.08 | 0.06 | 0.17 | 0.17 | 0.043 |
| Cholesteryl sulphate K | | 0.07 | 0.10 | 0.05 | 0.05 | 0.11 | | 0.040 |
| Cetylphosphate | 0.06 | | | | | | 0.08 | |
| Tocophersolan ® | 0.25 | | | 0.32 | | | | |
| (Eastman) | | | | | | | | |
| Cremophor ® EL | | 0.46 | 0.34 | 0.63 | 0.33 | 0.50 | | 0.32 |
| Solutol ™ HS-15 | | | | | | | 0.48 | |
| Tyloxapol ® | 0.20 | 0.53 | 0.54 | 0.00 | 0.65 | 1.07 | 1.08 | 0.30 |
| 50% Lecithin solution | | 0.82 | 0.63 | 0.55 | | 2.00 | 2.00 | |
| (Phospholipon ® S-80) | | | | | | | | |
| Lecithin hydrogenated | | | | | 0.70 | | | |
| 50% susp. | | | | | | | | |
| (Phospholipon ® S-80 | | | | | | | | |
| H) | | | | | | | | |
| AQUEOUS PHASE | | | | | | | | |
| Sodium citrate | 0.18 | 0.07 | 0.17 | 0.10 | 0.10 | 0.60 | 0.28 | 0.08 |
| (dihydrate) | | | | | | | | |
| Arginine base | | | | 0.07 | 0.07 | 0.90 | 0.30 | 0.10 |
| Water 60° C. | to 20 ml | to 20 ml | to 20 ml | to 20 ml | to 20 ml | to 40 ml | to 40 ml | to 20 ml |
| Filtration via 0.2-μm | ++ | + | − | −−− | ± | +++ | +++ | +++ |
| filter | | | | | | | | |
| Appearance after 2 | stable | separation | separation | separation | precipitate | stable | stable | stable |
| months at RT | | | | | | | | |

The preparation of polymixin formulations was carried out in a manner similar to that used in the previous examples, with some modification. The components of the lipid phase were mixed together, and heated to 60-65° C. using a water bath, while stirring with a spatula; hot water (60° C.) was then added. The resulting suspension was mixed for 30 minutes at 3,000-5,000 rpm, using a rotor-stator type high shear mixer (Omni GLH 115, USA). Samples were centrifuged (3000 rpm, 15 minutes) and filtrated through a 0.45-μm membrane filter.

Formulations of other antibiotics, such as vancomycin hydrochloride, capreomycin, colistin sulfate, ampicillin dihydrate, cephalosporin, levofloxacin, moxifloxacin, and gatifloxacin, were prepared in a manner similar to that used to prepare previously described formulations. Each prepared formulation had a drug content in the range of 2-50 mg/ml.

### Examples 52-59

TABLE 10
Solid lipid nanoparticles loaded with antibiotics and antibacterial compounds

| Components | Ex. 52 | Ex. 53 | Ex. 54 | Ex. 55 | Ex. 56 | Ex. 57 | Ex. 58 | Ex. 59 |
|---|---|---|---|---|---|---|---|---|
| LIPID PHASE | | | | | | | | |
| Tocopherol | 0.55 | 0.65 | 0.5 | 0.55 | 0.55 | 0.55 | 0.55 | 0.55 |
| succinate | | | | | | | | |
| Suppocire ® CM | 1.00 | 1.10 | 1.00 | 1.00 | 1.00 | 1.00 | 1.00 | 1.00 |
| Ampicillin | 0.12 | | | | | | | |
| trihydrate | | | | | | | | |
| Vancomycin HCl | | 0.24 | | | | | | |
| Colistin sulfate | | | 0.15 | | | | | |
| Capreomycin | | | | 0.10 | | | | |
| sulfate | | | | | | | | |
| Erythromycin | | | | | 0.12 | | | |
| base | | | | | | | | |
| Ofloxacin | | | | | | 0.12 | | |
| Cefoxitin free acid | | | | | | | 0.12 | |
| Moxifloxacin | | | | | | | | 0.16 |
| Cetylphosphate | 0.07 | | 0.08 | 0.04 | 0.08 | | | |
| Cholesteryl | | 0.10 | | | | 0.06 | | 0.06 |
| sulphate potassium | | | | | | | | |
| Tocophersolan ® | 0.35 | 0.40 | 0.35 | 0.35 | 0.35 | 0.35 | 0.35 | 0.35 |
| (Eastman) | | | | | | | | |
| Cremophor ® EL | 0.43 | 0.6 | 0.5 | 0.4 | 0.43 | 0.43 | | 0.43 |
| 50% Lecithin | | | | | | | | |
| solution | | | | | | | | |
| (Phospholipon ® S- | | | | | | | | |
| 80) | | | | | | | | |
| 1,2-Dipalmitoyl-sn- | | | | | | | 0.06 | |
| glycero-3- | | | | | | | | |
| ethylphospho- | | | | | | | | |
| choline chloride | | | | | | | | |
| AQUEOUS | | | | | | | | |
| PHASE | | | | | | | | |
| Sodium citrate | 0.18 | 0.26 | 0.60 | 0.14 | 0.18 | 0.20 | 0.24 | 0.25 |
| (anhydrous) | | | | | | | | |
| Arginine base | | | 0.40 | | | | | |
| Water 60° C. | to 20 ml | to 20 ml | to 20 ml | to 20 ml | to 20 ml | to 20 ml | to 20 ml | to 20 ml |
| Filtration via 0.2-μm | ++ | + | ++ | ++ | ++ | + | ++ | ++ |
| filter | | | | | | | | |
| Appearance | stable | stable | stable | stable | stable | stable | stable | stable |
| | suspension | suspension | suspension | suspension | suspension | suspension | suspension | suspension |

The particle size for the suspensions of Examples 52-59 was in the range of 100-450 nm (D50) and 380-1100 (D90). The resulting colloidal lipid formulations were stable. Antibiotic incorporated into the lipid colloidal delivery system maintained antibacterial activity, and could be used for the treatment of diseases caused by susceptible microorganisms. To provide long-tern storage, the colloidal formulations of the invention can be lyophilized using standard approaches and common lyophilization aids, including, for example, trehalose, lactose, sucrose, mannitol, glycine, polyvinylpyrrolidone, or dextran, in an appropriate ratio.

While the present invention has been described with reference to what is presently considered to be a preferred embodiment, it is to be understood that the invention is not limited to the disclosed embodiment. To the contrary, the invention is intended to cover various modifications and equivalent arrangements included within the spirit and scope of the appended claims.

All publications, patents, and patent applications are herein incorporated by reference in their entireties, to the same extent as if each individual publication, patent, or patent application was specifically and individually indicated to be incorporated by reference in its entirety.

We claim:

1. A drug carrier comprising a solid lipid nanoparticle (SLN), wherein the SLN comprises tocopherol or a derivative thereof.

2. The drug carrier of claim 1, wherein the SLN comprises a tocopherol ester.

3. The drug carrier of claim 2, wherein the tocopherol ester is selected from the group consisting of tocopheryl palmitate, tocopheryl stearate, tocopheryl behenate, tocopheryl succinate, tocopheryl phosphate, tocopheryl enantate, tocopheryl acetate, and tocopheryl nicotinate.

4. The drug carrier of claim 1, which is loaded with a biologically active compound.

5. The drug carrier of claim 4, wherein the biologically active compound is water soluble.

6. The drug carrier of claim 5, wherein the water-soluble biologically active compound is an antibiotic.

7. The drug carrier of claim 6, wherein the antibiotic is selected from the group consisting of an aminoglycoside, a macrolide, a polypeptide, a fluoroquinolone, a penicillin, and a cephalosporin.

8. The drug carrier of claim 6, wherein the antibiotic is selected from the group consisting of streptomycin, gentamicin, kanamycin, amikacin, neomycin, rifampicin, erythromycin, lincomycin, vancomycin, capreomycin, colistin, polymixin, gramicidin, ampicillin, cephalosporin, levofloxacin, moxifloxacin, and gatifloxacin.

9. The drug carrier of claim 1, further comprising a hydrophobic adjuvant.

10. The drug carrier of claim 9, wherein the hydrophobic adjuvant is a charged compound.

11. The drug carrier of claim 10, which is loaded with a water-soluble biologically active compound, wherein the hydrophobic adjuvant and the water-soluble biologically active compound have charged moieties of opposite signs.

12. A method for preparing the drug carrier of claim 1.

13. The method of claim 12, which does not use high-pressure homogenization.

14. The method of claim 13, which does not use an organic solvent.

15. A pharmaceutical composition comprising a solid lipid nanoparticle (SLN) and a biologically active compound, wherein the SLN comprises tocopherol or a derivative thereof.

16. The pharmaceutical composition of claim 15, wherein the biologically active compound is water soluble.

17. The pharmaceutical composition of claim 16, wherein the water-soluble biologically active compound is an antibiotic.

18. A colloidal drug delivery system comprising solid lipid nanoparticles (SLNs), wherein the SLNs comprise tocopherol or a derivative thereof.

19. The colloidal drug delivery system of claim 18, wherein the SLNs comprise a tocopherol ester.

20. The colloidal drug delivery system of claim 19, wherein the tocopherol ester is selected from the group consisting of tocopheryl palmitate, tocopheryl stearate, tocopheryl behenate, tocopheryl succinate, tocopheryl phosphate, tocopheryl enantate, tocopheryl acetate, and tocopheryl nicotinate.

21. The colloidal drug delivery system of claim 18, wherein at least some of the SLNs are loaded with a biologically active compound.

22. The colloidal drug delivery system of claim 21, wherein the biologically active compound is water soluble.

23. The colloidal drug delivery system of claim 22, wherein the water-soluble biologically active compound is an antibiotic.

24. The colloidal drug delivery system of claim 23, wherein the antibiotic is selected from the group consisting of an aminoglycoside, a macrolide, a polypeptide, a fluoroquinolone, a penicillin, and a cephalosporin.

25. The colloidal drug delivery system of claim 23, wherein the antibiotic is selected from the group consisting of streptomycin, gentamicin, kanamycin, amikacin, neomycin, rifampicin, erythromycin, lincomycin, vancomycin, capreomycin, colistin, polymixin, gramicidin, ampicillin, cephalosporin, levofloxacin, moxifloxacin, and gatifloxacin.

26. The colloidal drug delivery system of claim 21, wherein the colloidal drug delivery system has a lipid phase, and wherein at least 50% of the biologically active compound is associated with the lipid phase.

27. The colloidal drug delivery system of claim 21, which is capable of controlled delivery of the biologically active compound.

28. The colloidal drug delivery system of claim 27, wherein the delivery is via a parenteral, oral, nasal, pulmonary, rectal, topical, transdermal, or transmucosal route of administration.

29. The colloidal drug delivery system of claim 18, further comprising a hydrophobic adjuvant.

30. The colloidal drug delivery system of claim 29, wherein the hydrophobic adjuvant is a charged compound.

31. The colloidal drug delivery system of claim 30, wherein at least some of the SLNs are loaded with a water-soluble biologically active compound, and wherein the hydrophobic adjuvant and the water-soluble biologically active compound have charged moieties of opposite signs.

32. The colloidal drug delivery system of claim 18, further comprising a stabilizer selected from the group consisting of an ionic or non-ionic surfactant and a phospholipid.

33. A method of preparing the colloidal drug delivery system of claim 18.

34. The method of claim 33, wherein the method does not use high-pressure homogenization.

35. The method of claim 34, wherein the method does not use an organic solvent.
